(19) United States
(12) Patent Application Publication
(10) Pub. No.: US 2019/0216735 A1
(43) Pub. Date: Jul. 18, 2019

(54) LIPOSOMAL FORMULATIONS

(71) Applicants: NANYANG TECHNOLOGICAL UNIVERSITY, Singapore (SG); SINGAPORE HEALTH SERVICES PTE LTD, Singapore (SG)

(72) Inventors: Subramanian VENKATRAMAN, Singapore (SG); Yingying HUANG, Singapore (SG); Amrita NAGLE, Singapore (SG); Jayaganesh V. NATARAJAN, Singapore (SG); Nicolas Daniel Marie FOIN, Singapore (SG); Tze Tec CHONG, Singapore (SG); Jie Liang PHUA, Singapore (SG)

(21) Appl. No.: 16/332,725

(22) PCT Filed: Sep. 14, 2017
(86) PCT No.: PCT/SG2017/050461

§ 371 Date: Mar. 12, 2019

(30) Foreign Application Priority Data
Sep. 14, 2016 (SG) 10201607641Q

(51) Int. Cl. A61K 9/127; A61K 9/00; A61K 31/337; A61K 31/436; A61P 9/00; A61M 25/10; A61M 25/00

(52) U.S. Cl. CPC A61K 9/127; A61K 9/1278; A61K 9/0019; A61M 25/0084; A61K 31/436; A61P 9/00; A61M 25/104; A61K 31/337

(57) ABSTRACT

According to the present disclosure, a liposomal formulation comprising at least one uncharged phospholipid without cholesterol and at least one pharmaceutical agent, wherein the at least one uncharged phospholipid forms one or more lipid bilayers without cholesterol encapsulating the at least one pharmaceutical agent, is provided. The use(s) of such a liposomal formulation and method of producing such a liposomal formulation are also provided.

### CROSS-REFERENCE TO RELATED APPLICATION

This application claims the benefit of priority of Singapore Patent Application No. 10201607641Q, filed 14 Sep. 2016, the content of it being hereby incorporated by reference in its entirety for all purposes.

### TECHNICAL FIELD

The present disclosure relates to a liposomal formulation, its uses and method of producing such a liposomal formulation.

### BACKGROUND

Cardiovascular disease (CVD) is a general term for heart and blood vessel diseases including, but not limited to, atherosclerosis, coronary heart disease, cerebrovascular disease, aorto-iliac disease, and peripheral vascular disease. Subjects with CVD may develop a number of complications such as myocardial infarction, stroke, angina pectoris, transient ischemic attacks, congestive heart failure, aortic aneurysm, death etc.

CVD is one of the leading causes of death globally. According to the World Health Organization (WHO), it is responsible for about 31% of all deaths worldwide. CVD typically stems from vascular dysfunction, for example, as a result of atherosclerosis, thrombosis or high blood pressure, which then compromises organ function. Notably, the heart and brain may be affected, which tend to occur in myocardial infarction and stroke, respectively. 80% of CVD deaths are likely due to coronary heart disease and stroke according to the WHO. Two other types of CVD are peripheral arterial disease (PAD) and aortic disease. In the United States, it is estimated that 1.5 million Americans require an intervention (about 407,400 receive bypass or amputation; 1,080,000 receive angioplasty, stent or atherectomy) annually as a result of PAD.

In the past few decades, major improvements have been made in treating some types of CVD. For example, therapies such as angioplasty and insertion of stents have reduced death rates. In another example, in the treatment of coronary artery disease, the first generation of drug eluting stents (DESs) demonstrated reductions in restenosis. However, their long-term use has been marred by late stent thrombosis due to incomplete healing, especially after discontinuation of dual antiplatelet therapy.

Recently, adoption of new generation DES and their unrestricted use in clinical practice have proven that these devices may be more effective and safer compared to first generation DES. Nevertheless, DES are not immune to restenosis. In fact, routine angiographic surveillance after unrestricted use of new generation DES demonstrated rates of angiographic restenosis of approximately 12%.

Additionally, treatment of patients with DES-IRS (IRS: in-stent restenosis) has proven to be challenging.

Although the value of drug eluting balloon in de novo lesions remains controversial, they have been proven to be very effective in patients with both BMS-IRS (BMS: bare metal stent) and DES-IRS.

In the case of Patent ductus arteriosus (PDA), treatment with percutaneous endovascular intervention has emerged as a preferred alternative to amputation or surgery due to its relatively less invasiveness and shorter recovery periods for the patient. One of the primary goals of intervention is, therefore, to improve blood supply to leg muscles so that patients maintain their functional level of activity, especially for a claudicant.

In critical limb ischemia, the Below The Knee (BTK) vessels are particularly difficult to treat due to the need of maintaining flow to the more distal extremity to prevent amputation. Moreover, durability of this treatment may be limited by high rate of restenosis and re-interventions in this segment where, currently, about one-third of patients have a patent artery one year after intervention with angioplasty of the femoral artery. If either rest pain or tissue loss (gangrene or ulceration) sets in, revascularization may be a key method to limb preservation and amputation prevention for current clinical treatment.

Alternatively, BMSs have eliminated the elastic recoil associated with balloon angioplasty and flow-limiting dissections but insignificantly alleviate restenosis due to intimal hyperplasia. Unfortunately, stents placed in the femoro-popliteal segment provide patency rates of only 54-63% at 1 year and 28-55% at 2 years, and drug applied directly fails to eliminate restenosis.

Conventional drug delivery mechanisms, as well as treatment and prevention means as discussed above, suffer a limitation in that they tend to be restricted to addressing problems of CVD and not other medical conditions, such as cancer tumors, diseases of the pulmonary track and/or diseases of the gastrointestinal tracts.

There is thus a need to provide for a composition that resolves and/or ameliorates the issues mentioned above. The composition is at least deliverable to the target tissue directly without compromising sustained release of at least one pharmaceutical agent of the composition.

There is also a need to provide for a method of making such a composition, and the use of such a composition, to ameliorate one or more of the issues as mentioned above. The use of such a composition includes, but not limited to, a method of treating and/or preventing the diseases mentioned above or for manufacture of a medicament to treat and/or prevent the diseases mentioned above.

### SUMMARY

In one aspect, there is provided for a use of a liposomal formulation comprising at least one uncharged phospholipid without cholesterol and at least one pharmaceutical agent in the manufacture of a medicament for treatment and/or prevention of cardiovascular diseases, cancer tumors, diseases of pulmonary track and/or diseases of gastrointestinal tract, wherein the at least one uncharged phospholipid forms one or more lipid bilayers without cholesterol encapsulating the at least one pharmaceutical agent.

In another aspect, there is provided for a liposomal formulation comprising at least one uncharged phospholipid without cholesterol and at least one pharmaceutical agent for use in the treatment and/or prevention of cardiovascular diseases, cancer tumors, diseases of pulmonary track and/or diseases of gastrointestinal tract, wherein the at least one uncharged phospholipid forms one or more lipid bilayers without cholesterol encapsulating the at least one pharmaceutical agent.

In another aspect, there is provided for a method of treating and/or preventing cardiovascular diseases, cancer tumors, diseases of pulmonary track and/or diseases of gastrointestinal tract, comprising: administering a liposomal formulation comprising at least one uncharged phospholipid without cholesterol and at least one pharmaceutical agent to a target site, wherein the at least one uncharged phospholipid forms one or more lipid bilayers without cholesterol encapsulating the at least one pharmaceutical agent.

In another aspect, there is provided for a method of producing a liposomal formulation comprising at least one uncharged phospholipid without cholesterol and at least one pharmaceutical agent, wherein the at least one uncharged phospholipid forms one or more lipid bilayers without cholesterol encapsulating the at least one pharmaceutical agent, comprising: providing a solution comprising the at least one uncharged phospholipid without cholesterol in an organic solvent; heating the solution under reduced pressure to form a thin film; and contacting the thin film with a hydrating medium to form multilamellar vesicles of the liposomal formulation.

### BRIEF DESCRIPTION OF THE DRAWINGS

The drawings are not necessarily to scale, emphasis instead generally being placed upon illustrating the principles of the invention. In the following description, various embodiments of the present disclosure are described with reference to the following drawings, in which:

FIG. 1 shows a schematic diagram illustrating a method of producing a liposomal formulation comprising a pharmaceutical agent by thin-film hydration technique, according to embodiments disclosed herein.

FIG. 2a shows a liposome (20) loaded with drugs (22), according to embodiments disclosed herein. The drugs (22) are found within the bilayer formed by phospholipids (21) as shown. Specifically, the drugs (22) (i.e. the at least one pharmaceutical agent) are disposed in an empty space flanked by the at least one phospholipid of the one or more lipid bilayers.

FIG. 2b shows a liposome incorporating sirolimus in the bilayers of the liposome. Specifically, sirolimus, a non-limiting example of the at least one pharmaceutical agent, is disposed among and/or adjacent to one or more hydrophobic tail ends of the at least one phospholipid forming the one or more lipid bilayers.

FIG. 3 shows a plot of in vitro cumulative release study of paclitaxel (PTX) from egg phosphatidylcholine (EPC) liposomes (denoted by curve 3-a) and PTX from 1-palmitoyl-2-oleoyl-sn-glycero-3-phosphocholine (POPC) liposomes (denoted by curve 3-b), both obtained after extrusion.

FIG. 4 shows a plot of in vitro PTX release rate (released per day) from EPC liposomes (denoted by curve 4-a) and PTX from POPC liposomes (denoted by curve 4-b), both obtained after extrusion.

FIG. 5 shows a plot of in vitro dialysis (cumulative) release study of sirolimus (i.e. rapamycin) from 7.6 mol % sirolimus-loaded EPC liposomes, obtained after extrusion.

FIG. 6 shows a plot of in vitro mass release rate of sirolimus from 7.6 mol % sirolimus-loaded EPC liposomes, obtained after extrusion.

FIG. 7 shows a Malvern Zetasizer analysis of a drug loaded liposome according to embodiments described herein.

FIG. 8 is a plot showing the viability (%) of Human Fetal Artery Smooth Muscle Cells upon treatment with PTX liposomes (percentage calculated with respect to (wrt) T₀ viability). The effect of liposome loaded with PTX on the Human Fetal Artery Smooth Muscle Cells is demonstrated through this plot.

FIG. 9 shows 2 injection zones along a 40 mm segment (left image) and the post-injection stenting (right image) in a pig.

FIG. 10 illustrates both left carotid and left iliac arteries of a pig administered with sirolimus-loaded EPC liposomes, and a right carotid artery administered with saline. The sirolimus-loaded EPC liposomes prepared for animal study, as in this instance, may be called nanolimus (NL).

FIG. 11 illustrates the timeline for blood collection from the pigs.

FIG. 12 shows the in-stent segment (lumen of the stent) where analysis and detection of the sirolimus in the left carotid and left iliac arteries are carried out.

FIG. 13 shows the 28 day arterial drug level (in-stent). The median value is 406.55 ng/g and the mean is 599.66 ng/g.

FIG. 14 shows the systemic blood levels of sirolimus concentration for 1 hour, 24 hours and 28 days.

FIG. 15a shows a schematic diagram of the histology for analysis of the in-stent segments administered with either saline or NL.

FIG. 15b shows the histology of in-stent segments administered with saline (left image) and with NL (right image). The scale bars in both images are 1000 μm.

FIG. 15c shows the histology of various layers of the in-stent segments of FIG. 15b. The scale bars in both left and right images are 200 μm.

FIG. 16a shows the percentage of luminal stenosis, an indication of inflammation, for arterial segments administered with either saline or NL.

FIG. 16b shows the neointimal area, an indication of vascular injury, for arterial segments administered with either saline or NL.

FIG. 17a shows an image of mild restenosis in the right carotid artery.

FIG. 17b shows an image of total occlusion in-stent in left carotid artery.

FIG. 17c shows an image of moderate restenosis in left femoral/iliac artery.

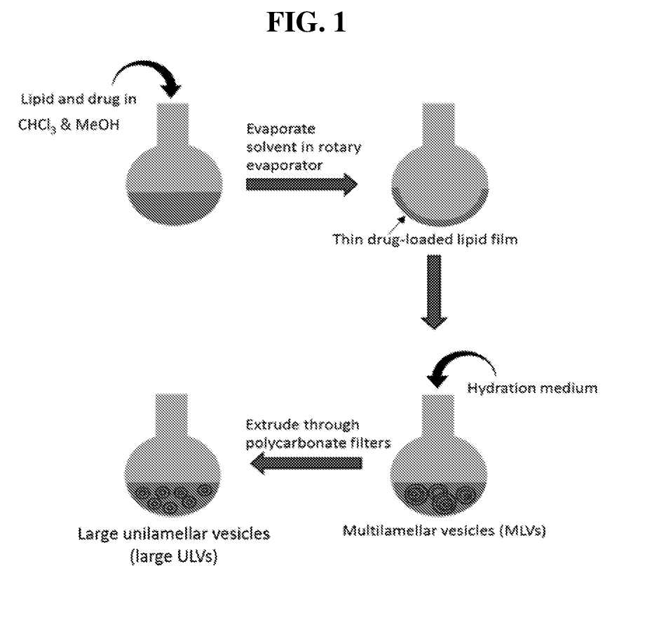

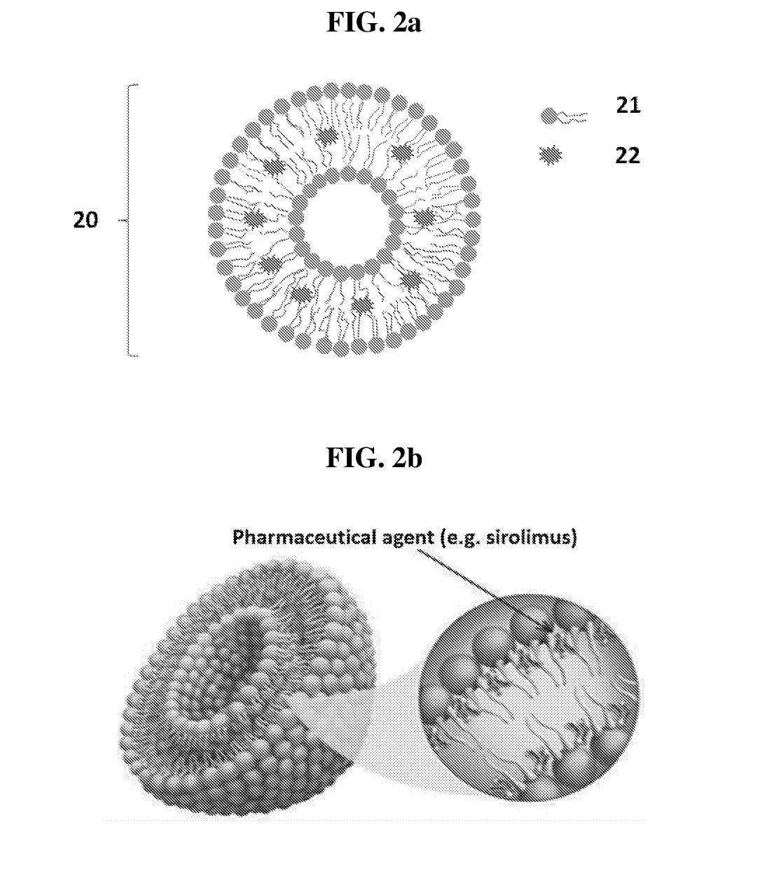

### DETAILED DESCRIPTION

The following detailed description refers to the accompanying drawings that show, by way of illustration, specific details and embodiments in which the invention may be practised.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

The present disclosure relates to use of a liposomal formulation, that does not contain cholesterol, in the manufacture of a medicament for treatment and/or prevention of various diseases, particularly cardiovascular diseases. The present disclosure also relates to such a liposomal formulation for use in the treatment and/or prevention of various diseases, particularly cardiovascular diseases. Such a liposomal formulation is advantageous for drug delivery.

In the present disclosure, the liposomal formulation may be called a pharmaceutical composition as it contains at least one pharmaceutical agent. The pharmaceutical agent may be a drug. The pharmaceutical agent may be present in the liposomal formulation in a pharmaceutically effective amount. The liposomal formulation may be called a liposomal composition, or concisely referred to as a formulation or a composition, in the present disclosure.

The liposomal formulation may include at least one uncharged phospholipid. Uncharged phospholipids are neutral phospholipids that do not contain any moiety having a charged group, i.e. either positive or negative, or they may be neutral because of having one or more moieties with a number of charged groups that neutralize each other to result in zero net charge. For example, an uncharged phospholipid may contain a positively charged group and a negatively charged group, such that the net charge of the phospholipid is zero. In the context of the present disclosure, an uncharged phospholipid, which is neutral, refers to a phospholipid that has a net charge of zero.

Based on the above, the liposomal formulation may include at least one uncharged phospholipid and at least one pharmaceutical agent present in a pharmaceutically effective amount. The at least one uncharged phospholipid may be a plurality of uncharged phospholipids forming at least one liposome entrapping (i.e. encapsulating) the at least one pharmaceutical agent.

Advantageously, the liposomal formulation is able to provide sustained release of the at least one pharmaceutical agent without the presence of cholesterol, i.e. without incorporating cholesterol in the liposomes. Cholesterol is conventionally used to make liposomes more rigid and/or resistant to diffusion, which helps to retain the liposome at a target site, or to retain the pharmaceutical agent(s) within the liposomes, for a period sufficient to provide sustained release of drugs. As opposed to such conventional use, the present liposomal formulation circumvents the use of cholesterol to achieve sustained release of drug(s).

The liposomal formulation is advantageously versatile in that it can be administered through various means. The liposomal formulation can be injected into a target tissue directly without use of any carriers. The liposomal formulation need not be administered intravenously. The liposomal formulation can be used together with a stent for providing sustained drug release to prevent e.g. future restenosis inside the stent. The liposomal formulation can also be used in the treatment of in-stent (i.e. lumen of stent) stenosis (e.g. re-narrowing) for an implanted stent. The liposomal formulation, being administrable to a subject in need thereof via the means as described above, is therefore advantageous for a method of treating and/or preventing various diseases, especially cardiovascular diseases.

The liposomal formulation and its various uses, also mitigate and/or avoid inflammation during its use and does not cause any vascular injury while enabling sustained release of pharmaceutical agent(s).

The present disclosure is further directed to a method of producing the liposomal formulation. The method allows the liposomal formulation to be produced without using any cholesterol. The present method also allows the amount of phospholipids to be portioned in relation to the drug so as to achieve a high encapsulation efficiency and retain the drug so as to provide sustained release. Through the present method, drug in the form of their crystals, i.e. drug crystal(s), need not be used to sustain release of drugs at or to a target site.

Having outlined various advantages of the present liposomal formulation, its uses and method of producing such a liposomal formulation, definitions of certain terms are first discussed before going into details of the various embodiments.

In the context of the present disclosure, the term “diameter” refers to the longest distance taken between two points on the external surface of a liposome (i.e. liposomal particle) measured through the center of the liposome.

In the context of the present disclosure, the phrase “organic solvent” refers to a liquid, or a mixture of liquids, that is carbon based and is capable of dissolving a phospholipid.

The word “substantially” does not exclude “completely” e.g. a composition which is “substantially free” from Y may be completely free from Y. Where necessary, the word “substantially” may be omitted from the definition of the invention.

In the context of various embodiments, the articles “a”, “an” and “the” as used with regard to a feature or element include a reference to one or more of the features or elements.

In the context of various embodiments, the term “about” or “approximately” as applied to a numeric value encompasses the exact value and a reasonable variance.

As used herein, the term “and/or” includes any and all combinations of one or more of the associated listed items.

As used herein, the phrase of the form of “at least one of A and B” may include A or B or both A and B. Correspondingly, the phrase of the form of “at least one of A and B and C”, or including further listed items, may include any and all combinations of one or more of the associated listed items.

Unless specified otherwise, the terms “comprising” and “comprise”, and grammatical variants thereof, are intended to represent “open” or “inclusive” language such that they include recited elements but also permit inclusion of additional, unrecited elements.

Having defined the various terms as mentioned above, details of the various embodiments are now described below.

In the present disclosure, there is provided for a use of a liposomal formulation comprising or consisting of at least one uncharged phospholipid without cholesterol and at least one pharmaceutical agent in the manufacture of a medicament for treatment and/or prevention of cardiovascular diseases, cancer tumors, diseases of pulmonary track and/or diseases of gastrointestinal tract, wherein the at least one uncharged phospholipid forms one or more lipid bilayers without cholesterol encapsulating the at least one pharmaceutical agent. The liposomal formulation advantageously provides sustainable and/or predictable dosing of the at least one pharmaceutical agent, for example, in a blood vessel to prevent and/or minimize stenosis or restenosis.

In various embodiments, the liposomal formulation may be composed of liposomes (i.e. liposomal particles). The liposomes may be completely spherical or substantially spherical. The liposomes may be multilamellar or unilamellar vesicles. The diameter of such vesicles may be 3 μm or less, 0.02 μm to 3 μm or 0.08 μm to 0.12 μm. For example, such vesicles may have an average diameter of 0.08 μm or 0.12 μm. Accordingly, the liposomal formulation may comprise liposomes having an average diameter of 3 μm or less, 0.02 μm to 3 μm or 0.08 μm to 0.12 μm. In some embodiments, the liposomes may have an average diameter of 0.02 μm to 3 μm. When the average diameter of the liposomes exceeds 3 μm, the liposomes administered, e.g. via injection, may have risk of obstructing the conduit of a micro-needle. Liposomes with average diameter of 3 μm or less tend to have lower risk of obstruction and allow less invasive (i.e. smaller sized) micro-needles to be used for administration.

The liposomes may be formed with one or more lipid bilayers. The lipid bilayers may be composed of a plurality of lipids, particularly phospholipids having zero net charge. Such neutral phospholipids are uncharged phospholipids as described above. The one or more lipid bilayers, advantageously circumvent the need for cholesterol to provide sustained release of drugs which it encapsulates. In other words, the one or more lipid bilayers do not contain cholesterol and the at least one phospholipid used to form the liposome does not contain cholesterol.

In various embodiments, the at least one pharmaceutical agent and the at least one uncharged phospholipid without cholesterol may have a molar ratio of 1:100 to 30:100. This means that the pharmaceutical agent (drug) may be present in an amount of 1 to 30 mole percent drug. Through portioning of phospholipids and pharmaceutical agent(s) alone, a liposomal formulation that provides sustained release of the pharmaceutical agent(s) without the presence of cholesterol is advantageously derived. Further advantageously, the pharmaceutical agent(s), i.e. the drug(s), need not be in their crystal form to achieve such sustained release. That is to say, the liposomal formulation contains no drug crystals or substantially no drug crystals.

In various embodiments, uncharged (neutral) lipids may include, but are not limited to, phosphatidylcholine (PC), phosphatidyl ethanolamine (PE), sphingomyelin, alkyl ether lecithin, and their combinations thereof. Such uncharged lipids may be used with other lipids. Accordingly, in various embodiments, the at least one uncharged phospholipid without cholesterol may be selected from the group consisting of phosphatidylcholine (PC), phosphatidyl ethanolamine (PE), sphingomyelin, alkyl ether lecithin, and combinations thereof.

In the context of the present disclosure, the term “phosphatidylcholine” generally refers to a class of phospholipids (amphipathic lipids) that incorporate choline as a head group with one or more phosphate groups attached to it. Such phosphatidylcholine (PC) may be selected from the group consisting of 1,2-dioleoyl-sn-glycero-3-phosphocholines (DOPC), 1,2-dioleoyl-sn-glycero-O-ethyl-3-phosphocholines, 1,2-dilauroyl-sn-glycero-3-phosphocholines (DLPC), 1,2-dimyristoyl-sn-glycero-3-phosphocholines (DMPC), 1,2-dipalmitoyl-sn-glycero-3-phosphocholines (DPPC), 1,2-distearoyl-sn-glycero-3-phosphocholines (DSPC), 1-palmitoyl-2-oleoyl-sn-glycero-3-phosphocholine (POPC), L-α-phosphatidylcholine or 95% egg phosphatidylcholine (EPC), and combinations thereof.

In some embodiments, the phosphatidylcholine may comprise or consist of POPC. In some embodiments, the phosphatidylcholine may comprise or consist of at least one unsaturated fatty acid moiety. For example, the phosphatidylcholine may comprise or consist of L-α-phosphatidylcholine or EPC.

As mentioned above, the at least one pharmaceutical agent, including its derivatives, may be encapsulated in liposomes of the present liposomal formulation. They may be sufficiently retained even without cholesterol in the liposomes' bilayer so that sustained release can be achieved at a target site.

In various embodiments, the at least one pharmaceutical agent may comprise or consist of an anti-proliferative drug such as paclitaxel, sirolimus and similar limus derivatives as well as mitomycin C, and similar agents. The present liposomal formulation enables sustained delivery of such pharmaceutical agent(s).

In various embodiments, the at least one pharmaceutical agent may be selected from the group consisting of anti-proliferative agents, anti-proliferative and antimitotic alkylating agents, anti-proliferative and antimitotic antimetabolites, platinum coordination complexes, hormones, non-steroidal agents, para-aminophenol derivatives, indole and indene acetic acids, heteroaryl acetic acids, arylpropionic acids, anthranilic acids, enolic acids, gold compounds, immunosuppressive agents, angiogenic agents, nitric oxide donors, anti-sense oligo nucleotides, and combinations thereof.

Anti-proliferative agents may include paclitaxel and epidipodophyllotoxins (e.g. etoposide and teniposide) etc.

Anti-proliferative and antimitotic alkylating agents may include nitrogen mustards (e.g. mechlorethamine, cyclophosphamide and its analogs, melphalan, and chlorambucil), ethylenimines, methylmelamines (e.g hexamethylmelamine and thiotepa), alkyl sulfonates-busulfan, nitrosoureas (e.g. carmustine (BCNU) and its analogs, and streptozotocin), trazenes-dacarbazinine (DTIC) etc.

Anti-proliferative and antimitotic antimetabolites may include folic acid analogs (e.g. methotrexate), pyrimidine analogs (e.g fluorouracil, floxuridine, and cytarabine), purine analogs and its related inhibitors (e.g. mercaptopurine, thioguanine, pentostatin, 2-chlorodeoxyadenosine {cladribine}) etc.

Platinum coordination complexes may include (e.g. cisplatin and carboplatin), procarbazine, hydroxyurea, mitotane, aminoglutethimide etc.

Hormones may include estrogen, adrenaline, progesterone, aldosterone, cortisol, glucagon, prolactin, luteinizing hormone etc.

Non-steroidal agents may include salicylic acid derivatives (e.g. aspirin), ibuprofen, naproxen, nabumetone etc.

Para-aminophenol derivatives may include acetaminophen, acetanilid, acetophenetidin etc.

Indole and indene acetic acids may include indomethacin, sulindac, etodalac etc.

Heteroaryl acetic acids may include tolmetin, diclofenac, ketorolac etc.

Arylpropionic acids may include ibuprofen, naproxen, flubiprofen and their derivatives etc.

Anthranilic acids may include mefenamic acid, meclofenamic acid, tolfenamic acid, flufenamic acid etc.

Enolic acids may include piroxicam, tenoxicam, phenylbutazone, oxyphenthatrazone etc.

Gold compounds may include auranofin, aurothioglucose, gold sodium thiomalate etc.

Immunosuppressive agents may include cyclosporine, tacrolimus (PK-506), sirolimus (rapamycin), azathioprine, mycophenolate mofetil etc.

Angiogenic agents may include vascular endothelial growth factor (VEGF), fibroblast growth factor (FGF), platelet-derived growth factor (PDGF), Factor VIII, interleukin 8 (IL-8) etc.

Nitric oxide donors may include arginine, nitroprusside, nitroglycerin etc.

Anti-sense oligo nucleotides may include eteplirsen, volanesorsen, nusinersen, mipomersen, morpholino, fomivirsen etc.

Deoxyribonucleic acid (DNA) or ribonucleic acid (RNA), their derivatives, and their combinations thereof, may be used as the pharmaceutical agent(s) as well.

The pharmaceutical agent(s) listed above, or any other pharmaceutical agent(s) used, may be a drug, that is not in the crystal form. Advantageously, the at least one pharmaceutical agent may be sustainably released through the liposomal formulation without the need to be in crystal form.

Advantageously, the present liposomal formulation is usable in the manufacture of a medicament for treating and/or preventing various diseases. The diseases include cardiovascular diseases, cancer tumors, diseases of pulmonary track and/or diseases of gastrointestinal tract.

Cardiovascular diseases may be selected from the group consisting of atherosclerosis, coronary heart disease, cerebrovascular disease, aorto-iliac disease, peripheral vascular disease, venous disease, structural heart disease, cardiomyopathies, chronic total occlusion, acute myocardial infarction, restenosis and chronic limb ischemia.

Cancer tumors may be selected from the group consisting of non-small cell lung cancer, lymphoblastic leukemia and ovarian cancer.

Diseases of pulmonary track may be selected from the group consisting of lung cancer, cystic fibrosis and chronic obstructive pulmonary disease (COPD).

Diseases of gastrointestinal tract may be selected from the group consisting of Crohn's disease, ulcerative colitis, gastric cancer and colon cancer.

The medicament manufactured based on the present liposomal formulation may be used in drug delivery for treatment or passivation of unstable plaque, wherein the medicament (containing a pharmaceutical agent) may be administered by injection into the vessel wall. The medicament may also be used to deliver drug into the adventitia of arterial vessels for targeting nerve(s), by means of stimulation or inhibition of the nerve(s). Such nerve(s) may comprise or consist of sympathetic and/or parasympathetic nerve(s) known for modulating blood pressure control.

The present disclosure also provides for a liposomal formulation comprising or consisting of at least one uncharged phospholipid without cholesterol and at least one pharmaceutical agent for use in the treatment and/or prevention of cardiovascular diseases, cancer tumors, diseases of pulmonary track and/or diseases of gastrointestinal tract, wherein the at least one uncharged phospholipid forms one or more lipid bilayers without cholesterol encapsulating the at least one pharmaceutical agent.

The embodiments and advantages described above in relation to use of the liposomal formulation may be applicable and/or valid to embodiments of the liposomal formulation, and vice versa.

As described above, the liposomal formulation may comprise liposomes having an average diameter of 3 μm or less, 0.02 m to 3 μm or 0.08 μm to 0.12 μm. In some embodiments, the liposomes may have an average diameter of 0.02 μm to 3 μm. Liposomes with average diameter of 3 μm or less are less likely to get stuck in a micro-needle and are compatible with less invasive (smaller sized) micro-needles.

As described above, the at least one pharmaceutical agent and the at least one uncharged phospholipid without cholesterol may have a molar ratio of 1:100 to 30:100. This means that the pharmaceutical agent (drug) may be present in an amount of 1 to 30 mole percent drug. Through portioning of phospholipids and pharmaceutical agent(s) alone in forming the liposomal formulation, sustained release of pharmaceutical agent(s) without the presence of cholesterol is advantageously achieved. Further advantageously, the pharmaceutical agent(s), i.e. the drug(s), need not be in their crystal form to achieve such sustained release. That is to say, the liposomal formulation contains no drug crystals or substantially no drug crystals.

As described above, the at least one uncharged phospholipid without cholesterol may be selected from the group consisting of phosphatidylcholine (PC), phosphatidyl ethanolamine (PE), sphingomyelin, alkyl ether lecithin, and combinations thereof. The phosphatidylcholine (PC) may be selected from the group consisting of 1,2-dioleoyl-sn-glycero-3-phosphocholines (DOPC), 1,2-dioleoyl-sn-glycero-O-ethyl-3-phosphocholines, 1,2-dilauroyl-sn-glycero-3-phosphocholines (DLPC), 1,2-dimyristoyl-sn-glycero-3-phosphocholines (DMPC), 1,2-dipalmitoyl-sn-glycero-3-phosphocholines (DPPC), 1,2-distearoyl-sn-glycero-3-phosphocholines (DSPC), 1-palmitoyl-2-oleoyl-sn-glycero-3-phosphocholine (POPC), L-α-phosphatidylcholine or 95% egg phosphatidylcholine (EPC), and combinations thereof. These phospholipids advantageously form one or more lipid bilayers without cholesterol, thereby circumventing the need for cholesterol to provide sustained release of drugs that the lipid bilayer(s) encapsulates.

As described above, the at least one pharmaceutical agent that may be encapsulated by liposomes of the liposomal formulation may be selected from the group consisting of anti-proliferative agents, anti-proliferative and antimitotic alkylating agents, anti-proliferative and antimitotic antimetabolites, platinum coordination complexes, hormones, non-steroidal agents, para-aminophenol derivatives, indole and indene acetic acids, heteroaryl acetic acids, arylpropionic acids, anthranilic acids, enolic acids, gold compounds, immunosuppressive agents, angiogenic agents, nitric oxide donors, anti-sense oligo nucleotides, and combinations thereof. Non-limiting examples of these pharmaceutical agents have been listed above.

The pharmaceutical agent(s) as described in the present disclosure, or any other pharmaceutical agent(s), may be a drug, that is not in the crystal form. Advantageously, the at least one pharmaceutical agent may be sustainably released through the liposomal formulation without the need to be in crystal form.

Cardiovascular diseases, cancer tumors, diseases of pulmonary track and diseases of gastrointestinal tract, which are treatable and/or preventable by the liposomal formulation, are already listed above.

The liposomal formulation may also be used to treat and/or prevent other diseases and medical conditions that have been described above or elsewhere in the present disclosure.

The present disclosure further provides for a method of treating and/or preventing cardiovascular diseases, cancer tumors, diseases of pulmonary track and/or diseases of gastrointestinal tract, comprising: administering a liposomal formulation comprising at least one uncharged phospholipid without cholesterol and at least one pharmaceutical agent to a target site, wherein the at least one uncharged phospholipid forms one or more lipid bilayers without cholesterol encapsulating the at least one pharmaceutical agent.

Embodiments and advantages described above in relation to the liposomal formulation and its use, may be applicable and/or valid to embodiments pertaining to method(s) of treating and/or preventing the various diseases using the liposomal formulation and the medicament derived from such a liposomal formulation, and vice versa. For instance, one advantage of the present method is its capability to provide sustained release of the at least one pharmaceutical agent that is encapsulated by the liposomal formulation without involving cholesterol. The present method also does not require the pharmaceutical agent(s) to be in its crystal form to achieve such effect.

In various embodiments, the step of administering may comprise injecting the liposomal formulation directly to the target site by micro infusion catheter injection. By administering the liposomal formulation via injection, for example, into the adventitial layer, sustained and predictable dosing of the at least one pharmaceutical agent in the blood vessel wall may be achieved, thereby preventing and/or minimizing stenosis or restenosis.

In various embodiments, the step of administering may also comprise applying the liposomal formulation in a vessel layer or through a vessel wall (e.g. into the peri-adventitial space of a vessel) by balloon angioplasty with or without a stent. This approach improves, inter alia, vessel patency without causing severe inflammation or vascular injury, especially after percutaneous balloon angioplasty. In embodiments where a stent may be used, the present method may be used to limit restenosis if the stent is expanded in the vessel. The stents may be expandable without aid or may be expanded by balloon deployment. Accordingly, the method may comprise administering the liposomal formulation by combining with balloon or stent to provide sustained release of the at least one pharmaceutical agent.

Other embodiments of the method may include treatment or passivation of unstable plaque, by injection of the present liposomal formulation, encapsulating a pharmaceutical agent, into the vessel wall.

The method may also be used in drug delivery and administration into the adventitia of arterial vessels for targeting nerve(s), by means of stimulation or inhibition of the nerve(s). In some embodiments, such nerve(s) may comprise or consist of sympathetic and/or parasympathetic nerve(s) known to modulate blood pressure control.

In other embodiments of the present method, the pharmaceutical agent may be a therapeutic agent comprising or consisting of DNA and/or RNA, and their derivatives, to treat and/or prevent the various diseases mentioned in the present disclosure.

In the present method, the liposomal formulation may be administered or applied to a target site that includes, but is not limited to, a vessel layer, a peri-adventitial space of a vessel, an adventitial layer and/or a vessel wall.

Non-limiting examples of the at least one uncharged phospholipid and the at least one pharmaceutical agent have been described above.

Non-limiting examples of cardiovascular diseases, cancer tumors, diseases of pulmonary track and diseases of gastrointestinal tract, which are treatable and/or preventable by the present method, have been listed above. Apart from these diseases, the present liposomal formulation (e.g. its medicament) and the present method may also be used to treat and/or prevent other diseases and medical conditions.

A method of producing a liposomal formulation comprising at least one uncharged phospholipid without cholesterol and at least one pharmaceutical agent, wherein the at least one uncharged phospholipid forms one or more lipid bilayers without cholesterol encapsulating the at least one pharmaceutical agent is provided in the present disclosure.

Embodiments and advantages described above in relation to the liposomal formulation and its use, including the methods of treatment and/or prevention involving such liposomal formulation, may be applicable and/or valid to embodiments pertaining to method(s) of producing such a liposomal formulation, and vice versa.

The production method may comprise: providing a solution comprising or consisting of the at least one uncharged phospholipid without cholesterol in an organic solvent; heating the solution under reduced pressure to form a thin film; and contacting the thin film with a hydrating medium to form multilamellar vesicles of the liposomal formulation.

According to various embodiments, the providing step may comprise dissolving the at least one pharmaceutical agent in the organic solvent. Non-limiting examples of the at least one pharmaceutical agent have been described above. This step of dissolving may be preferably performed for pharmaceutical agent(s) that is not water soluble. The organic solvent may comprise chloroform, dichloromethane, cyclohexane, acetone and/or an alcohol. The alcohol may be methanol, ethanol, isopropanol or tert-butanol. The organic solvent may solely contain chloroform or an alcohol. The organic solvent may also be a mixture of liquids, for example, chloroform and an alcohol. In some instances, the organic solvent may be a mixture of chloroform and methanol.

Subsequently, the solution may be subjected to heating and, optionally, stirring. The heating and, optionally stirring, may be carried out in a water bath at a temperature of 30° C. to 65° C. The heating and, optionally stirring, may be carried out in a rotary evaporator at 50 to 200 rotations per minute (rpm). The heating and, optionally stirring, may also be carried out under reduced pressure. In some embodiments, vacuum may be applied during heating to achieve reduced pressure. This implies that the heating and, optionally stirring, may be carried out in vacuum in some instances. Heating the solution, whether under reduced pressure or entirely vacuum, helps to evaporate the organic solvent to accelerate formation of the thin film.

The method may further comprise a step of dissolving the at least one pharmaceutical agent in the hydrating medium prior to contacting the thin film with the hydrating medium instead of dissolving the at least one pharmaceutical agent in the organic solvent. This may be especially the case when the pharmaceutical agent(s) is water soluble, which may be added at this stage of production instead of during or before the step of providing the solution comprising the organic solvent. The hydrating medium may comprise a buffer solution, distilled water and/or non-electrolytes solutions. Buffer solution may include phosphate buffers, saline buffers etc. Non-electrolytes solutions may include sugar, saline solutions etc. The hydrating medium may comprise or consist of phosphate buffer solution, hydroxyethyl piperazine ethane sulfonic acid-Hanks' balanced salt solution (HEPES-HBSS), distilled water and/or non-electrolytes solutions. The phosphate buffer solution may be a phosphate buffered saline.

After the above steps, liposomes encapsulating the at least one pharmaceutical agent may be formed. The liposomes may exist as multilamellar vesicles (MLVs) as described above. The MLVs may be downsized by extrusion through a filter or by sonication. Accordingly, the production method may further comprise a step of extruding or sonicating the multilamellar vesicles to obtain unilamellar vesicles (ULVs) of the desired size. ULVs have been described above.

In some embodiments, the at least one pharmaceutical agent may be encapsulated after the liposomes are formed but before conversion to ULVs. The pharmaceutical agent(s) may also be encapsulated after the liposomes are converted to ULVs. In other words, the method may further comprise a step of contacting the MLVs or ULVs with the at least one pharmaceutical agent, in order to encapsulate the latter into the MLVs or ULVs.

While the methods described above are illustrated and described as a series of steps or events, it will be appreciated that any ordering of such steps or events are not to be interpreted in a limiting sense. For example, some steps may occur in different orders and/or concurrently with other steps or events apart from those illustrated and/or described herein. In addition, not all illustrated steps may be required to implement one or more aspects or embodiments described herein. Also, one or more of the steps depicted herein may be carried out in one or more separate acts and/or phases.

### EXAMPLES

The present disclosure relates to liposomal formulations for treatment and/or prevention of cardiovascular diseases, which includes specific treatment of stenosis, restenosis and in-stent stenosis of blood vessel, where the blood vessel includes coronary artery, peripheral artery and the vessel of the Below The Knee (BTK). The liposomal formulation may be referred to as a liposomal composition, a pharmaceutical composition, a formulation or a composition.

The composition includes one or more uncharged phospholipids and one or more anti-proliferative drugs, wherein the drug to lipid ratio may be 1:100 to 30:100 (1 to 30 mol % drug).

The liposomal formulation disclosed herein may be composed of liposomes (i.e. liposomal particles) having a size of 0.02 μm to 3 μm. The size may refer to average diameter.

The present formulation is administrable by micro infusion catheter injection to provide sustained release of the at least one pharmaceutical agent (e.g. anti-proliferative drug(s)) for at least 2 to 4 weeks.

The lipids used in the present disclosure belong to a class of uncharged neutral lipids, specifically a class of phospholipids (amphipathic lipids) that has choline as a head group with one or more phosphate groups attached to it. At the opposing end constitutes a hydrophobic tail group. Non-limiting examples of such phospholipids include, 1-palmitoyl-2-oleoyl-sn-glycero-3-phosphocholine (POPC), and L-α-phosphatidylcholine or 95% egg phosphatidylcholine (EPC), which may be used alone or in combination.

Meanwhile, the anti-proliferative drug(s) may include, but is not limited to, paclitaxel and analogs of sirolimus (i.e. rapamycin and derivatives thereof).

The present disclosure also relates to uses of the present liposomal formulation. For instance, the present liposomal formulation or pharmaceutical composition may be administered by micro infusion catheter injection to provide sustained release of one or more pharmaceutical agents. By applying the liposomal formulation via injection into an adventitial layer, a predictable and sustained dosing of the vessel wall can be attained.

The present disclosure further provides for a method of producing the present liposomal formulation, encapsulating at least one pharmaceutical agent, by thin-film hydration technique. Thin-film hydration technique enables uniform encapsulation of drug(s).

The technique of thin-film hydration generally refers to a technique that may be performed by firstly dissolving basic components forming a liposome membrane in an organic solvent such as chloroform, secondly subjecting the solution to a rotary evaporator to distill off the solvent by heating under reduced pressure to form a thin film on the inner side of the evaporator, and thirdly hydrating the thin film with a phosphate buffer solution, or a hydroxyethyl piperazine ethane sulfonic acid-Hanks' balanced salt (HEPES-HBSS) solution, in a warm water bath. When the drug is water-soluble, it may be dissolved in an aqueous solution used for hydration of the thin film, and when the drug is water-insoluble, it may be dissolved in an organic solvent together with the liposome-forming components. When the drug is water-soluble, it can be encapsulated after the liposomes are formed. That is to say, the method may include a step of adding the drug(s) to the liposomes for encapsulation after the latter is formed.

In various embodiments, the method may further comprise downsizing the liposomes by extrusion through a filter or by sonication. Water-soluble drug(s) may also be added to the downsized liposomes at this stage for encapsulation.

As an example of thin-film hydration technique, the required quantities of egg phosphatidylcholine (EPC) or 1-palmitoyl-2-oleoyl-sn-glycero-3-phosphocholine (POPC) and anti-proliferative drug may be weighed and dissolved in a mixture of chloroform and methanol at a drug to lipid mole ratio of 1:100 to 20:100. The drug-lipid mixture-containing flask may be attached to a rotary evaporator and immersed halfway in a water bath set at 40° C. A thin film may be then obtained by evaporating the organic solvent mixture while rotating the flask at 150 rotations per minute (rpm). The lipid thin film may then be hydrated with phosphate buffered saline (PBS at 150 mM, pH 7.4) to form multilamellar vesicles (MLVs). The MLVs may be further extruded through filters to get liposomes of the desired size.

The present liposomal formulation, its uses and method of production, are described in the examples below.

### Example 1: Preparation of Drug-Loaded Liposomes

The liposomal formulations as described herein were prepared by the thin-film hydration technique.

As a non-limiting example, quantities of egg phosphatidylcholine (EPC) or 1-palmitoyl-2-oleoyl-sn-glycero-3-phosphocholine (POPC) and paclitaxel required to make a 20 mM solution were weighed and dissolved in a 2:1 v/v (2 ml:1 ml) mixture of chloroform:methanol at a drug to lipid molar ratio of 0.05:1. The drug-lipid mixture-containing flask was attached to a rotary evaporator and immersed halfway in a water bath set at 40° C. A thin film was obtained by evaporating the organic solvent mixture while rotating the flask at 150 rpm. The lipid film was hydrated with phosphate buffered saline (PBS at 150 mM, pH 7.4) to form multilamellar vesicles (MLVs). The MLVs were extruded through polycarbonate filters (0.2 m and/or 0.08 m) to yield large unilamellar vesicles (large ULVs) with sizes ranging between 0.08 μm to 0.12 μm.

### Example 2a: Characterization and Results of Drug-Loaded Liposomes—Size Stability Study

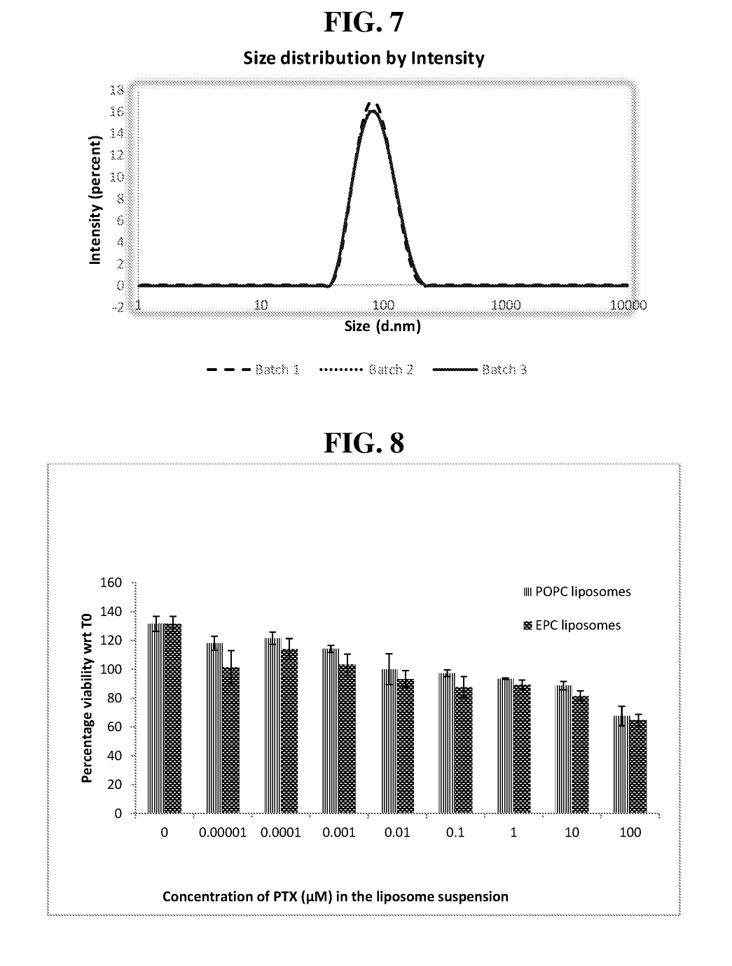

The mean size and size distribution of the liposomal particles were measured using Zetasizer Nano ZS (Malvern Instruments, Malvern, UK) that used dynamic light scattering to estimate size of particles in the nanometer range. The size of the liposomes (3 batches) was measured immediately after preparation and monitored upon storage at 4° C. Results are shown in FIG. 7.

The particle sizes of both the EPC and POPC liposomes were stable for at least up to 4 weeks at 4° C. (see table 1 below) and throughout the duration of the in-vitro release study for EPC.

TABLE 1
Particle size of liposomes upon preparation and during storage
at 4° C.

Average Particle Size,
nm (Polydispersity Index)

| Type of Liposomes | Upon Preparation | 4 Weeks (4° C.) |
|---|---|---|
| EPC | 94.97 (0.082) | 95.93 (0.113) |
| POPC | 91.81 (0.039) | 91.89 (0.186) |

### Example 2b: Characterization and Results of Drug-Loaded Liposomes—Drug Loading Efficiency

The amount contained in the liposomal suspension was determined by adding acetonitrile to a known amount of liposomes. These samples were vortexed and then centrifuged (13000 rpm for 15 minutes), and the supernatant was analyzed for drug content.

High loading efficiency (about 100%) was achieved with a drug to lipid molar ratio of 0.05 for both EPC and POPC liposomes. The final drug concentration in the liposomal suspension was 0.9 mg/ml for the EPC liposomes and 1 mg/ml for the POPC liposomes. The final drug to lipid weight percentage was 5.7% and 6.2% for EPC and POPC liposomes, respectively.

### Example 2c: Characterization and Results of Drug-Loaded Liposomes—In Vitro Drug Release Study

The liposomal suspension (1 ml) was placed in a cellulose ester bag (MWCO 100 kDa, 1.6 cm diameter by 5 cm length) and was dialyzed against 30 ml of release medium (PBS at pH 7.4 containing 10% v/v acetonitrile). The dialysis assembly was incubated at 37° C. in an orbital shaker at 50 rpm. Release samples were collected every 24 hours and the entire release medium was replaced with fresh medium to maintain dynamic sink conditions. Drug to lipid molar ratio was 0.05 for both types of liposomes.

Paclitaxel was quantified using an Agilent Series 1100 HPLC (Santa Clara, Calif.) connected to a UV-Vis detector, autosampler and column heater set at 35° C. The aliquots collected were filtered through a 0.2 m PTFE syringe filter into HPLC vials. Samples were analyzed using a ZORBAX Eclipse XDB-C18 (5 m) column with a mobile phase of acetonitrile:water 60:40 (volume ratio) at a flow rate of 1 ml/min, eluting the paclitaxel at about 5.6 minutes, with the UV-Vis detector recording the absorbance at 227 nm.

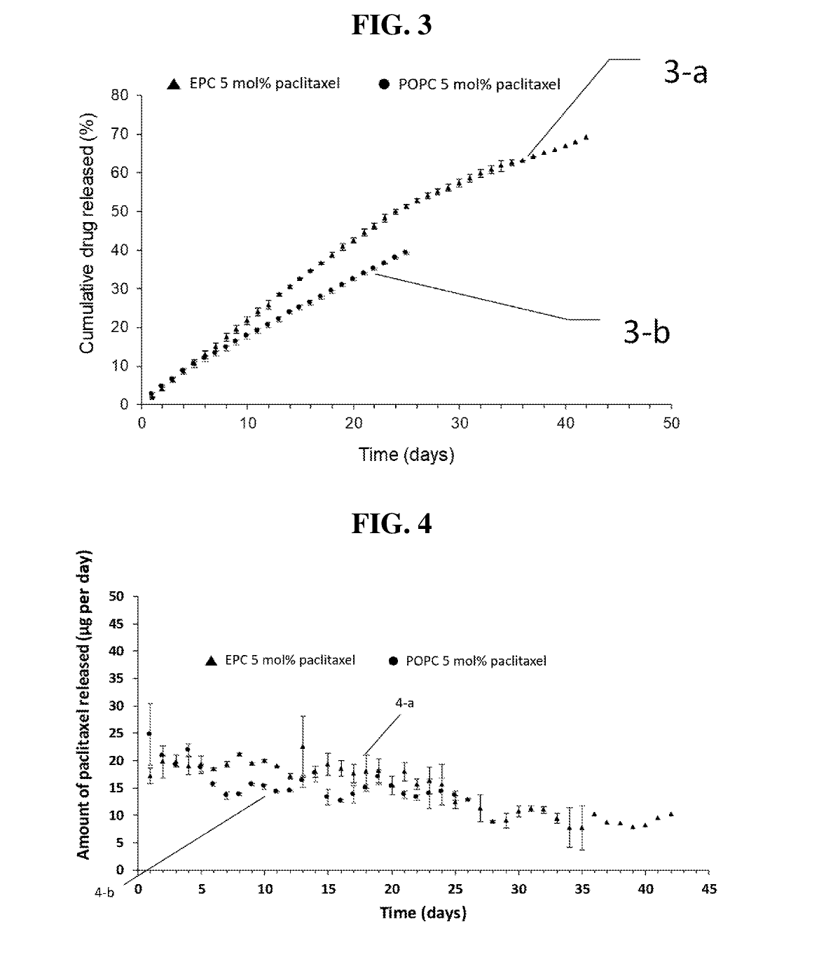

Release of paclitaxel from the liposomes was evaluated using the dialysis method. Drug release was sustained for more than 5 weeks. Cumulative percentage drug released over time is shown in FIG. 3 while amount of drug release per day is shown in FIG. 4. FIG. 3 shows the cumulative percentage drug released over time from EPC liposomes and POPC liposomes, denoted by curves 3-a and 3-b, respectively. FIG. 4 shows the amount of drug (i.e paclitaxel) released per day by EPC and POPC liposomes, denoted by curves 4-a and 4-b, respectively.

### Example 2d: Characterization and Results of Drug-Loaded Liposomes—Cytotoxicity Studies

The cytotoxicity of the liposomal formulation was tested against human fetal artery smooth muscle cells (hFSMCs). The cells were maintained in DMEM high glucose medium supplemented with 10% v/v fetal bovine serum (FBS), 100 units/ml penicillin and 1 μg/ml streptomycin at 37° C. and 5% CO₂. For the cytotoxicity assay, cells were plated in 96-well black plates with transparent bottom (Greiner 655090) at a seeding density of 10,000 cells per well. A few wells in a “T₀ plate” were also seeded at the same density. Following overnight incubation, cells were treated with the PTX-loaded liposomal formulations for 24 hours. Prestoblue cell viability reagent (Thermo Scientific) was added to the wells and the fluorescence was measured (Ex: 560 nm, Em: 590 nm) after 1 hour incubation at 37° C. For the T₀ wells, prestoblue reagent was added and viability was estimated at the time of drug addition to the treatment wells. Percentage viability of treated cells was obtained by comparing the fluorescence values against those of T₀ wells (T₀ represents viability at the time of treatment; taken as 100% viability). Viability (%) of hFSMCs is shown in FIG. 8.

### Example 3a: A Non-Limiting Exemplary Embodiment—28 Day Animal Study (Using Pigs) to Assess Drug Uptake and Efficacy

A further study was carried out in pigs to test the present liposomal formulation. Before the study, EPC liposomes encapsulating sirolimus were characterized.

The sirolimus liposomal suspension (150 μl) was placed in a cellulose ester bag (MWCO 100 kDa, 1 cm diameter by 5 cm length) and was dialyzed against 30 ml of release medium (PBS at pH 7.4). The dialysis assembly was incubated at 37° C. in an orbital shaker at 50 rpm. Entire release medium was replaced daily with fresh medium to maintain dynamic sink conditions. Residual analysis was performed at 3 hours, day 1, day 2, day 5, day 7, day 10, day 14, day 21 and day 28, by methanol extraction.

Sirolimus was quantified using reverse phase-HPLC of Agilent 1100 Series, coupled to a UV-Vis detector, auto-sampler and column heater set at 25° C. Samples were filtered through a 0.22 μm PTFE syringe filter into HPLC vials before being analyzed using an Agilent Eclipse 5 μm, 4.6×250 mm XDB C-18 column with an isocratic mobile phase consisting of methanol:water 85:15 (volume ratio) at a flow rate of 1 ml/min, eluting the sirolimus at about 7 minutes, with the UV-Vis detector recording the absorbance at 278 nm.

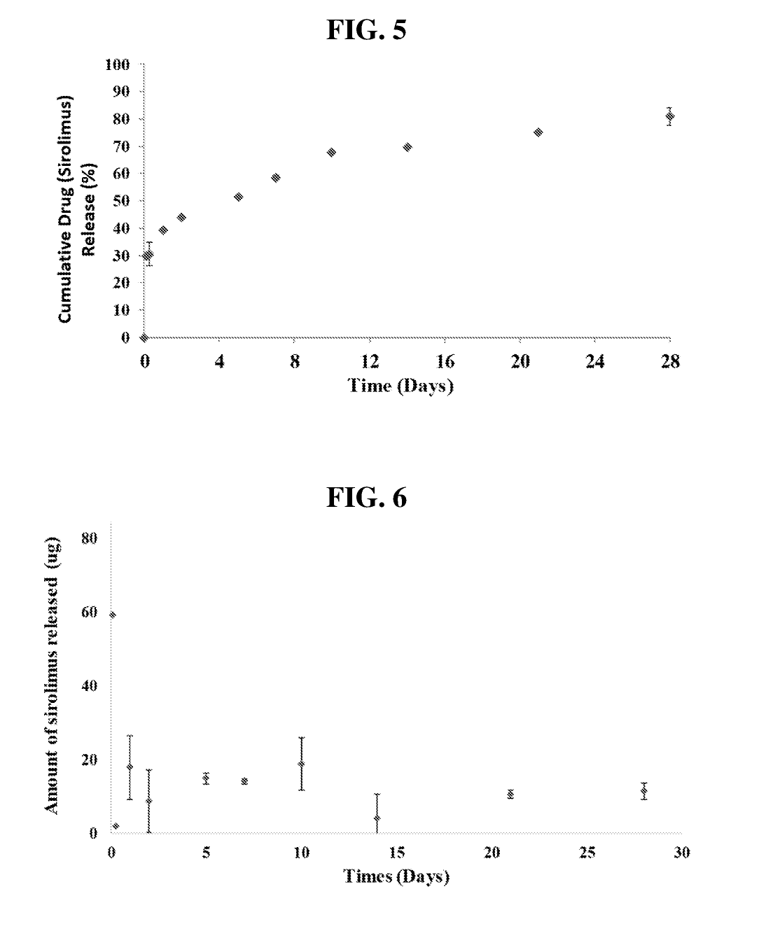

The EPC liposomes with sirolimus were about 80.17 nm±1.08 nm and their in vitro cumulative drug (i.e. sirolimus) release profile is similar to that as shown in FIG. 5. After characterization, these EPC liposomes were administered to the pigs.

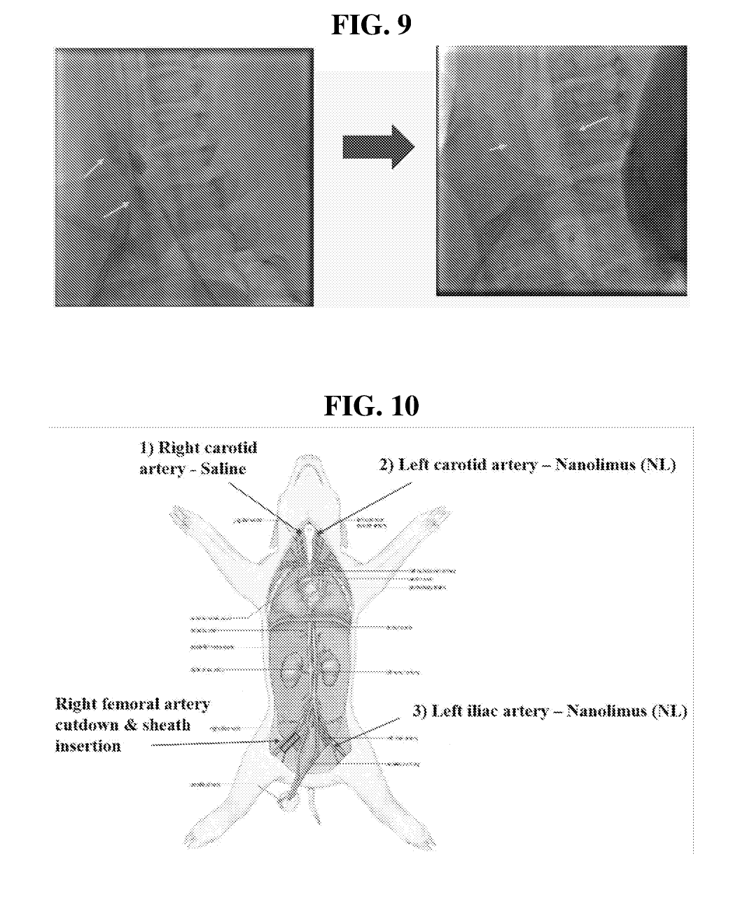

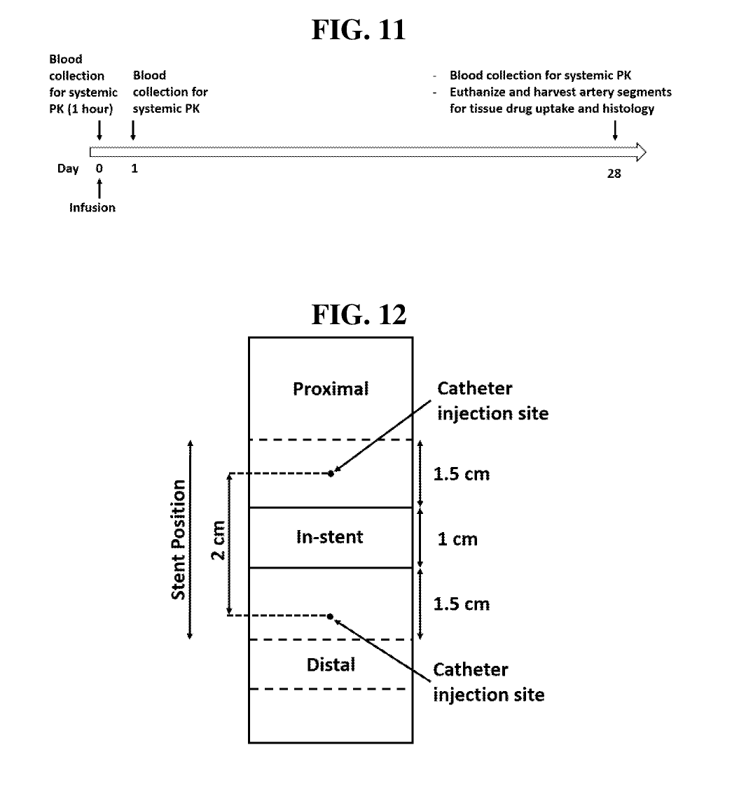

Administration of the EPC liposomes to pigs (or other animals) can be carried out via micro infusion catheter injection (Bullfrog catheter from Mercator MedSystems), as used in this example. The drug (i.e. sirolimus) was thus administered in the form of sirolimus encapsulated in EPC liposomes, which are referred to in this example as nanolimus (NL). 2 arteries per pig for 4 pigs were utilized for NL infusion and 1 artery in each of the 4 pigs was used for saline (FIG. 10). That is to say, a total of 8 arteries were administered with NL and a total of 4 arteries were administered with saline. 1 mg of sirolimus (as NL) was applied to each artery where 2 injection sites (FIG. 9) along each artery were made (0.467 mg/ml). The timeline for blood collection from the pigs at the 24^{th} hour and on the 28^{th} day is shown in FIG. 11.

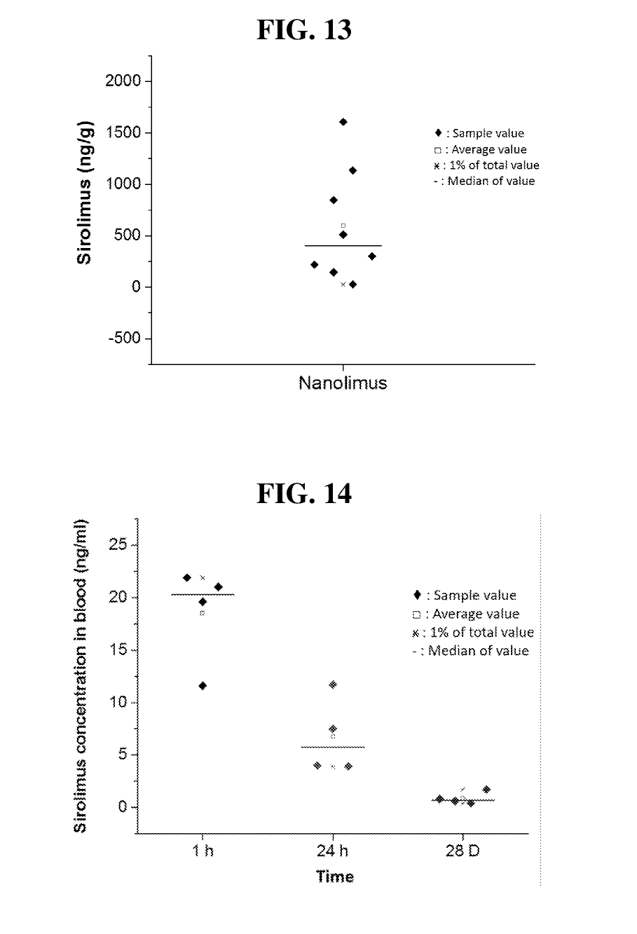

Analysis of sirolimus levels in the stented region of left carotid and left iliac arteries (middle of stent, 2 overlapping injections) were performed (FIG. 12). Significant levels of sirolimus from NL were detectable at day 28. From FIG. 13, better sustained release of sirolimus was demonstrated by the present liposomal formulation (NL in this instance) compared to reported studies using conventional nanoparticle albumin-bound rapamycin over the same period of 28 days.

TABLE 2
Sirolimus drug level in neointima (for in-stent arterial
segments)

| Artery | Arterial | Pig 1 | Pig 2 | Pig 3 | Pig 4 |
|---|---|---|---|---|---|
| Type | Segments | (NL) | (NL) | (NL) | (NL) |
| Left | Tunica media + | 27.5 | 131.3 | 529.4 | 771.8 |
| Carotid | adventitial | | | | |
| Artery | Neointima | — | 169.7 | 607.2 | 835.4 |
| Left Iliac | Tunica media + | 51.7 | 96.1 | 423.5 | 232.7 |
| Artery | adventitial | | | | |
| | Neointima | 94.3 | 123.8 | 423.5 | 279.4 |

The systemic blood levels of sirolimus in each pig for 1 hour, 24 hours and 28 days after infusion are shown in FIG. 14 for the four pigs administered with NL. No adverse events and no toxicity were observed in the pigs.

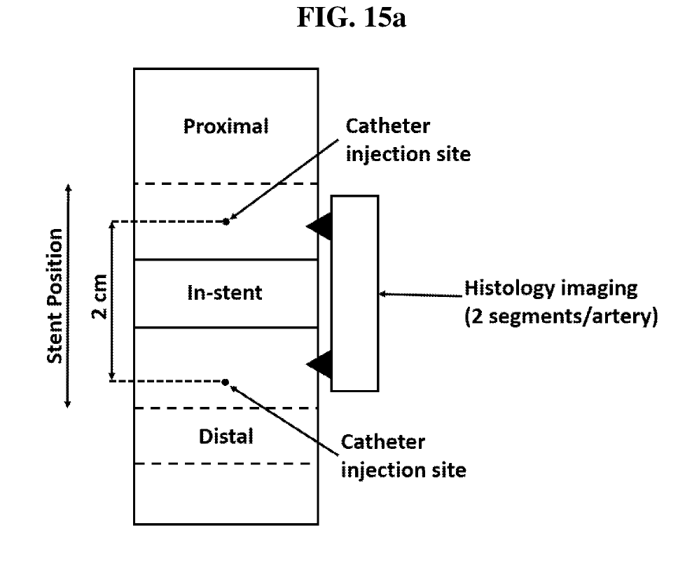

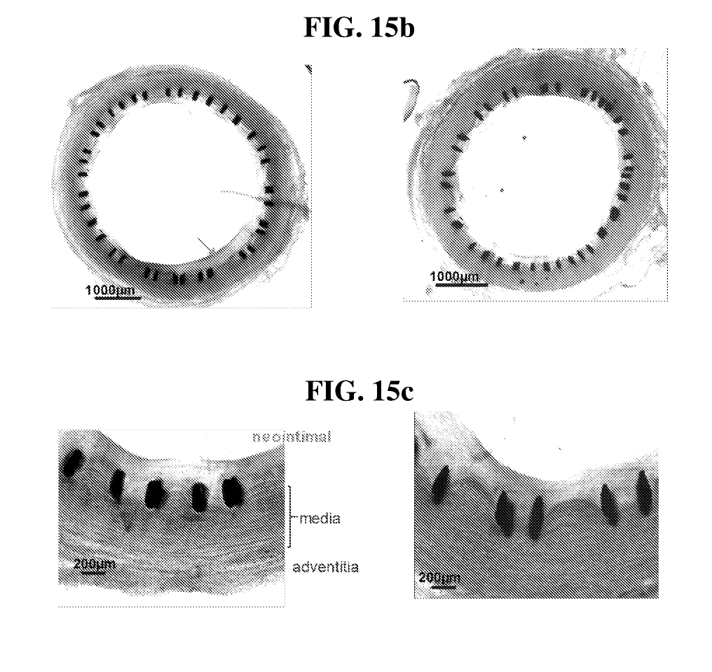

FIG. 15a to FIG. 15c show the histology of in-stent segments of artery administered with either saline or NL. FIG. 15a shows the histology section of artery. FIG. 15b shows the overview images of stented artery at week 4 with the detailed histology of the various layers shown in FIG. 15c, wherein FIG. 15c shows 3 layers of cells including the neointimal, the media and the adventitia layer.

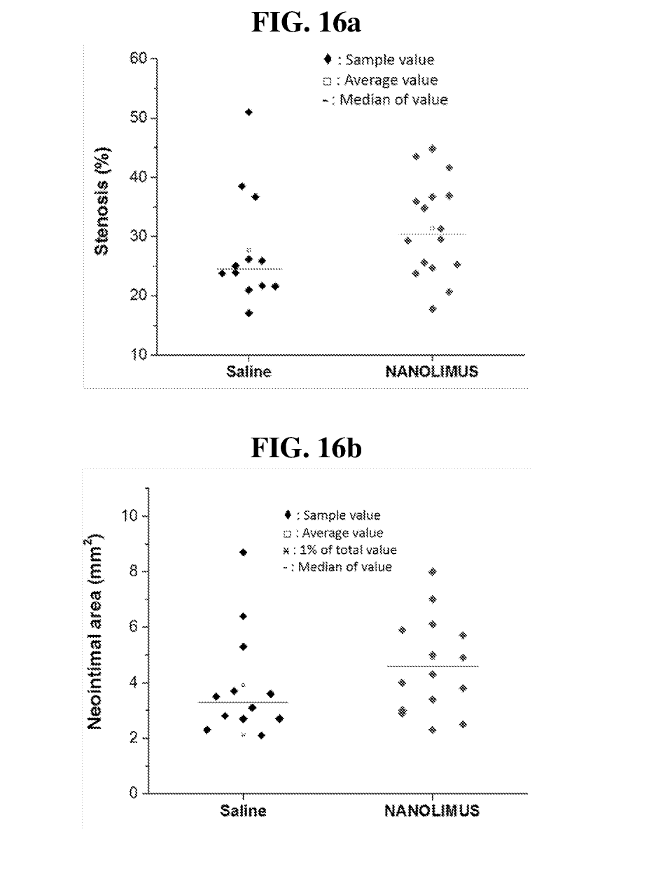

The histomorphometry results are shown in FIG. 16a and FIG. 16b. FIG. 16a shows the percentage of luminal stenosis, an indication of inflammation, for arterial segments administered with either saline or NL. No significant difference between the segments administered with saline and NL were observed. Table 3a below shows an inflammation scoring table and an inflammation scoring of less than 1 was observed for all segments.

TABLE 3a
Inflammation Scoring Table

| Score | Inflammation Severity |
|---|---|
| 0 | No inflammation cell near strut |
| 1 | Light, non-circumferential inflammatory infiltrate near strut |
| 2 | Localized, moderate to dense inflammatory aggregate near strut |
| 3 | Circumferential dense inflammatory infiltrate of the strut |

FIG. 16b shows neointimal area, an indication of vascular injury, for arterial segments administered with either saline or NL. No significant difference between the neointimal area administered with saline and NL were observed. Table 3b below shows the vascular scoring chart and a vascular injury score of 0 was observed for all segments.

TABLE 3b
Vascular Injury Scoring Table

| Score | Vascular Injury Severity |
|---|---|
| 0 | Intact internal elastic lamina |
| 1 | Laceration of internal elastic lamina |
| 2 | Laceration until media layer |
| 3 | Laceration until external elastic lamina/adventitial layer |

Out of the 12 arteries that were studied, only 1 artery, which was the first artery injected with saline, suffered in-stent thrombosis. Hence, the results clearly demonstrate better stenosis and imply reduced restenosis for the present liposomal formulation and its uses.

### Example 3b: A Non-Limiting Exemplary Embodiment—Follow-Up Study of Stenosis Model from Example 3a (Day 36)

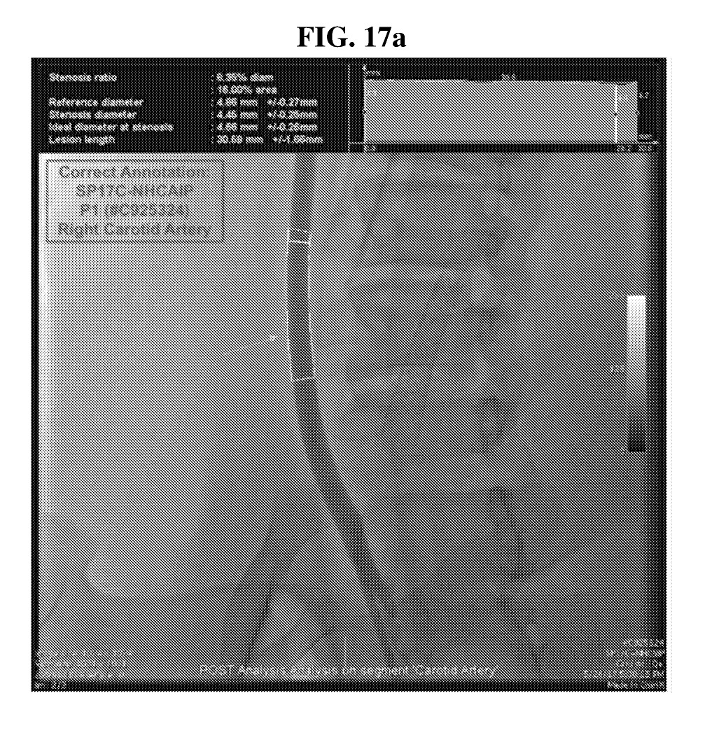

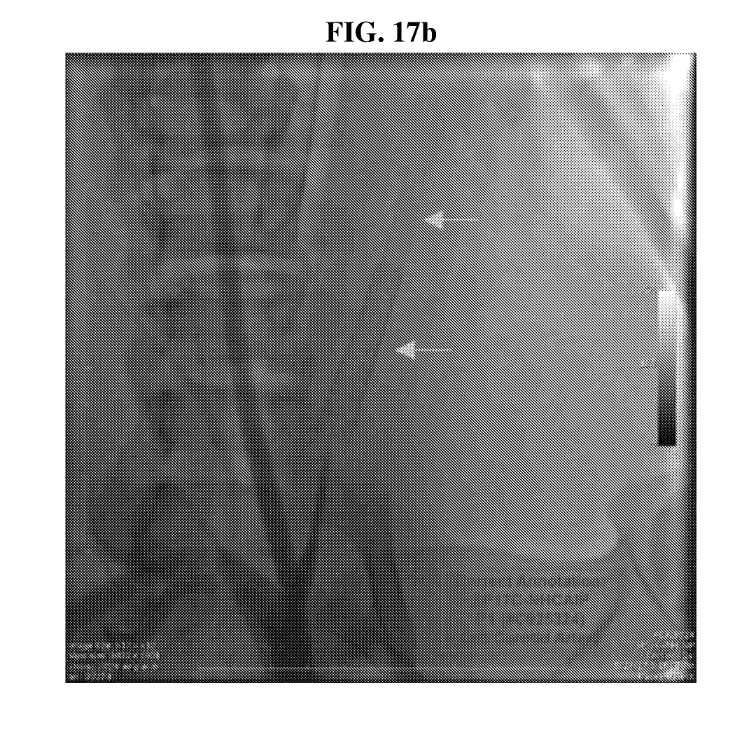

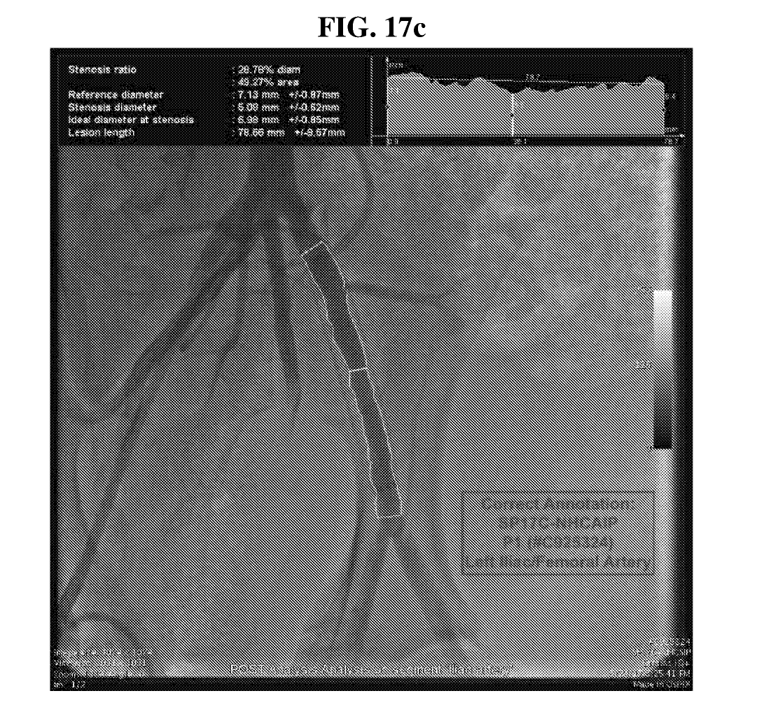

To attain a better restenosis model in pigs, the various administration methods were also studied. This is a separate study from the preceding example. The results were obtained from the 36^{th} day of the experiments of example 3a. In summary, the method represented by FIG. 17a, produced mild stenosis which was insufficient while the method represented by FIG. 17b was too serious, causing total occlusion. The method represented by FIG. 17c resulted in about 30% stenosis, which is likely ideal in subsequent model creation/studies.

According to FIG. 17a, mild restenosis (8.35% diameter, 16% area) was observed in right carotid artery for a method of balloon overexpansion by 3 times (3×) with denudation [denoted as (1) in this example].

According to FIG. 17b, total occlusion in-stent was observed in left carotid artery for a method including (1), use of balloon expandable stent [denoted as (2) in this example] and use of balloon [denoted as (3) in this example]. The final actual stent:artery ratio was 0.95:1. It is to be noted that 2 stents due to dissection created distal to target injury site.

According to FIG. 17c, moderate restenosis (28.78% diameter, 49.27% area) was observed in left femoral/iliac artery for a method including (1), a self-expandable stent and (3). Final actual stent:artery ratio was 1.1:1.

Based on the above, better restenosis model has been developed accordingly.

Further, based on all examples illustrated above, it has been shown that sustained drug release is achieved with the present liposomal formulation, for instance, the NL of examples 3a and 3b. This translates to higher arterial drug levels.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1-12. (canceled)

13. A liposomal formulation comprising at least one uncharged phospholipid without cholesterol and at least one pharmaceutical agent, wherein the at least one uncharged phospholipid forms one or more lipid bilayers without cholesterol encapsulating the at least one pharmaceutical agent.

14.-15. (canceled)

16. The liposomal formulation according to claim 13, wherein the at least one pharmaceutical gent and the at least one uncharged phospholipid without cholesterol having a molar ratio of 1:100 to 30:100.

17. The liposomal formulation according to claim 13, wherein the at least one uncharged phospholipid without cholesterol is selected from the group consisting of phosphatidylcholine (PC), phosphatidyl ethanolamine (PE), sphingomyelin, alkyl ether lecithin, and combinations thereof.

18. (canceled)

19. The liposomal formulation according to claim 13, wherein the at least one pharmaceutical agent is selected from the group consisting of anti-proliferative agents, anti-proliferative and antimitotic alkylating agents, anti-proliferative and antimitotic antimetabolites, platinum coordination complexes, hormones, non-steroidal agents, para-aminophenol derivatives, indole and indene acetic acids, heteroaryl acetic acids, arylpropionic acids, anthranilic acids, enolic acids, gold compounds, immunosuppressive agents, angiogenic agents, nitric oxide donors, anti-sense oligo nucleotides, and combinations thereof.

20. The liposomal formulation according to claim 13, wherein the at least one pharmaceutical agent is not in the crystal form.

21.-24. (canceled)

25. A method of treating and/or preventing cardiovascular diseases, cancer tumors, diseases of pulmonary track, and/or diseases of gastrointestinal tract, comprising:
administering a liposomal formulation comprising at least one uncharged phospholipid without cholesterol and at least one pharmaceutical agent to a target site, wherein the at least one uncharged phospholipid forms one or more lipid bilayers without cholesterol encapsulating the at least one pharmaceutical agent.

26. The method according to claim 25, wherein administering the liposomal formulation comprises:
injecting the liposomal formulation directly to the target site by micro infusion catheter injection; or
applying the liposomal formulation in a vessel layer or through a vessel wall by balloon angioplasty with or without a stent.

27. The method according to claim 25, wherein the target site is a vessel layer, a peri-adventitial space of a vessel, an adventitial layer, and/or a vessel wall.

28. A method of producing a liposomal formulation comprising at least one uncharged phospholipid without cholesterol and at least one pharmaceutical agent, wherein the at least one uncharged phospholipid forms one or more lipid bilayers without cholesterol encapsulating the at least one pharmaceutical agent, comprising:
providing a solution comprising the at least one uncharged phospholipid without cholesterol in an organic solvent;
heating the solution under reduced pressure to form a thin film; and
contacting the thin film with a hydrating medium to form multilamellar vesicles of the liposomal formulation.

29. The method according to claim 28, wherein the organic solvent comprises chloroform, dichloromethane, cyclohexane, acetone, and/or an alcohol.

30. The method according to claim 29, wherein the alcohol is methanol, ethanol, isopropanol, or tert-butanol.

31. The method according to claim 28, wherein the heating is carried out in a water bath at a temperature of 30° C. to 65° C.

32. The method according to claim 31, wherein the heating is carried out in a rotary evaporator at 50 to 200 rotations per minute (rpm).

33. The method according to claim 28, wherein providing the solution further comprises dissolving the at least one pharmaceutical agent in the organic solvent.

34. The method according to claim 28, further comprising dissolving the at least one pharmaceutical agent in the hydrating medium prior to contacting the thin film with the hydrating medium.

35. The method according to claim 28, wherein the hydrating medium comprises phosphate buffer solution, hydroxyethyl piperazine ethane sulfonic acid-Hanks' balanced salt solution (HEPES-HBSS), distilled water, and/or non-electrolytes solutions.

36. The method according to claim 28, further comprising extruding or sonicating the multilamellar vesicles to obtain unilamellar vesicles.

37. The method according to claim 36, further comprising contacting the multilamellar vesicles or unilamellar vesicles with the at least one pharmaceutical agent.

38. The method according to claim 25, wherein the at least one pharmaceutical agent and the at least one uncharged phospholipid without cholesterol are present in a molar ratio ranging from 1:100 to 30:100, and the at least one pharmaceutical agent is not in a crystal form.

39. The method according to claim 28, wherein the at least one pharmaceutical agent and the at least one uncharged phospholipid without cholesterol are present in a molar ratio ranging from 1:100 to 30:100, and the at least one pharmaceutical agent is not in a crystal form.
